(19) United States
(12) Patent Application Publication
(10) Pub. No.: US 2017/0296589 A1
(43) Pub. Date: Oct. 19, 2017

(54) COMPOSITIONS AND METHODS FOR TREATING LUNG REMODELING DISEASES

(71) Applicant: Case Western Reserve University, Cleveland, OH (US)

(72) Inventors: Tracey L. Bonfield, Cleveland, OH (US); Arnold I. Caplan, Clevleand, OH (US)

(21) Appl. No.: 15/513,703

(22) PCT Filed: Sep. 23, 2015
(86) PCT No.: PCT/US2015/051682

§ 371 Date: Mar. 23, 2017

Related U.S. Application Data
(60) Provisional application No. 62/053,860, filed on Sep. 23, 2014.

(51) Int. Cl. A61K 35/28; G01N 33/50; C12N 5/0775

(52) U.S. Cl. CPC A61K 35/28; C12N 5/0662; G01N 33/5073

(57) ABSTRACT

In one aspect, the present disclosure can relate to a method for treating a lung remodeling disease in a subject. One step of the method can include assaying one or more mesenchymal stem cells (MSCs) for one or more of the following effects: (1) reduce collagen deposition in the lungs of the subject; (2) increase expression of a transcription factor in an alveolar macrophage of the subject; (3) decrease expression of a cytokine in an alveolar macrophage of the subject; (4) decrease expression of a toll-like receptor (TLR) in an alveolar macrophage of the subject; (5) decrease mRNA synthesis of collagen 1 and/or collagen 3 in the subject; and (6) decrease the level of hyaluronic acid in the subject. A therapeutically effective amount of the one or more assayed MSCs found to have one or more of effects (1)-(6) can then be administered to the subject.

### RELATED APPLICATION

This application claims the benefit of U.S. Provisional Patent Application No. 62/053,860, filed Sep. 23, 2014, the entirety of which is hereby incorporated by reference for all purposes.

### TECHNICAL FIELD

The present disclosure relates generally to compositions and methods useful for treatment of lung remodeling diseases and, more particularly, to using mesenchymal stem cells having a desired potency to affect inflammation while at the same time decreasing remodeling and improving pulmonary function.

### BACKGROUND

Lung remodeling diseases (LRD) are a group of disorders that lead to progressive loss of function in the alveoli. Although the disease typically involves an initial acute inflammatory response, many patients do not seek treatment until the disease has progressed to a more advanced chronic phase. LRDs may be due to a number of different underlying factors. Exposure to occupational or environmental inhalants, including inhalation of organic dust, inorganic dust, cigarette smoke or noxious gases can often result in LRD. First line treatment requires identification and removal of the causative agent from the patient's environment. LRD can also be caused by exposure to certain drugs or ionizing radiation, as may occur during chemo- or radiation therapy in cancer patients. LRD may also result from an exaggerated immune response, such as in sarcoidosis, or part of a more systemic collagen vascular disorder. In many cases, the underlying cause of LRD remains unknown.

Although the initiating agent(s) or circumstances may vary, the immunopathogenic response of lung tissues generally involves a similar course of events. The initial response is inflammation of the air spaces and alveolar walls, causing an acute alveolitis. If the condition persists, inflammation spreads to the interstitium and vasculature of the alveoli. At early stages, the alveolar and adjacent capillary endothelial cells become leaky, leading to alveolar and septal edema, and the number of immune cells found in bronchoalveolar lavage (BAL) fluid and/or sputum increases. In particular, the number of polymorphonuclear leukocytes (PMNs), which normally comprise about 1-3% of the cellular component of BAL and/or sputum, can increase to 20% or more. Persistence in the inflammatory response leads to desquamation of the wall of the alveoli and compensatory proliferation of fibroblast in the interstitium. The resultant scarring of lung tissue leads to significant alterations in gas exchange and ventilatory function. LRD can also involve the bronchioles, and patients may present with bronchiolitis.

Current treatment options for LRD are limited and do not provide long-term improvement in most patients. Corticosteroids such as prednisone are often provided to reduce the inflammation associated with LRD. However, immunosuppressant therapy can lead to increased risk of infection in the compromised lung and a worsening of the condition with additional complications.

### SUMMARY

In one aspect, the present disclosure can relate to a method for treating a lung remodeling disease in a subject. One step of the method can include assaying one or more mesenchymal stem cells (MSCs) for one or more of the following effects: (1) reduce collagen deposition in the lungs of the subject; (2) increase expression of a transcription factor in an alveolar macrophage of the subject; (3) decrease expression of a cytokine in an alveolar macrophage of the subject; (4) decrease expression of a toll-like receptor (TLR) in an alveolar macrophage of the subject; (5) decrease mRNA synthesis of collagen 1 and/or collagen 3 in the subject; (6) decrease the level of hyaluronic acid in the subject; (7) decrease the number of macrophages in lung tissue of the subject; (8) decrease the number of eosinophils in lung tissue of the subject; (9) decrease the number of polymorphonuclear leukocytes (PMNs) in lung tissue of the subject; (10) decrease systemic levels of an antibody in the subject; (11) decrease weight loss in the subject; and (12) decrease epithelial cell hyperplasia in the subject. A therapeutically effective amount of the one or more assayed MSCs found to have one or more of effects (1)-(12) can then be administered to the subject.

In another aspect, the present disclosure can relate to an in vitro method for obtaining MSCs with a desired potency for one or more of the following: (1) reduce collagen deposition in the lungs of the subject; (2) increase expression of a transcription factor in an alveolar macrophage of the subject; (3) decrease expression of a cytokine in an alveolar macrophage of the subject; (4) decrease expression of a TLR in an alveolar macrophage of the subject; (5) decrease mRNA synthesis of collagen 1 and/or collagen 3 in the subject; (6) decrease the level of hyaluronic acid in the subject; (7) decrease the number of macrophages in lung tissue of the subject; (8) decrease the number of eosinophils in lung tissue of the subject; (9) decrease the number of polymorphonuclear leukocytes (PMNs) in lung tissue of the subject; (10) decrease systemic levels of an antibody in the subject; (11) decrease weight loss in the subject; and (12) decrease epithelial cell hyperplasia in the subject. The method can include assaying a preparation of MSCs for, and selecting, MSCs with a desired potency for one or more of the effects (1)-(12) above.

In another aspect, the present disclosure can relate to a composition comprising MSCs assayed and selected for a desired potency to achieve one or more of the following effects: (1) reduce collagen deposition in the lungs of the subject; (2) increase expression of a transcription factor in an alveolar macrophage of the subject; (3) decrease expression of a cytokine in an alveolar macrophage of the subject; (4) decrease expression of a TLR in an alveolar macrophage of the subject; (5) decrease mRNA synthesis of collagen 1 and/or collagen 3 in the subject; (6) decrease the level of hyaluronic acid in the subject; (7) decrease the number of macrophages in lung tissue of the subject; (8) decrease the number of eosinophils in lung tissue of the subject; (9) decrease the number of polymorphonuclear leukocytes (PMNs) in lung tissue of the subject; (10) decrease systemic levels of an antibody in the subject; (11) decrease weight loss in the subject; and (12) decrease epithelial cell hyperplasia in the subject.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is an image showing that intravenously administered human mesenchymal stem cells (hMSCs) localize in the lungs of asthma mice;

FIG. 2 is a graph showing cellular differentiation associated with a chronic asthma response;

FIG. 3 is a graph showing that hMSCs decrease total cell recruitment in the context of induced chronic lung inflammation;

FIG. 4 is a graph showing that hMSCs improve inflammation in chronic asthma mice;

FIGS. 5A-C show hMSC treatment decreased lung pathology associated with the chronic asthma model;

FIGS. 6A-C are series of graphs showing plethysmography in the chronic ovalbumin murine model of asthma; and

FIGS. 7A-B are a series of graphs showing soluble (FIG. 7A) and insoluble collagen (FIG. 7B) changes with hMSCs.

### DETAILED DESCRIPTION

It should be understood that the present disclosure is not limited to the particular methodology, protocols, and reagents, etc., described herein and, as such, may vary. The terminology used herein is for the purpose of describing particular aspects only, and is not intended to limit the scope of the present disclosure.

The section headings are used herein for organizational purposes only and are not to be construed as in any way limiting the subject matter described.

The methods and techniques of the present disclosure are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane, Antibodies: A Laboratory Manual, Cold Spring Harbor Laboratory Press, Cold Spring Harbor, N.Y. (1990).

### Definitions

Unless otherwise defined, all technical terms used herein have the same meaning as commonly understood by one of ordinary skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of and similar phrases using words of the root “comprise” are used herein as synonyms of “comprising” and have the same meaning. “Comprised of” is a synonym of “comprising”.

As used herein, the term “and/or” can include any and all combinations of one or more of the associated listed items.

As used herein, the term “cell bank” can refer to industry nomenclature for cells that have been grown and stored for future use. Cells may be stored in aliquots. They can be used directly out of storage or may be expanded after storage. This is a convenience so that there are “off the shelf” cells available for administration. The cells may already be stored in a pharmaceutically-acceptable excipient so they may be directly administered or they may be mixed with an appropriate excipient when they are released from storage. Cells may be frozen or otherwise stored in a form to preserve viability. In one aspect of the present disclosure, cell banks are created in which the cells have been selected for enhanced potency to achieve one or more of certain effects, described below. Following release from storage, and prior to administration to a subject, it may be preferable to again assay the cells for potency. This can be done using any of the assays, direct or indirect, described in this application or otherwise known in the art. Then cells having the desired potency can then be administered to the subject for treatment.

As used herein, the term “co-administer” can refer to administration in conjunction with one another, together, coordinately, including simultaneous or sequential administration of two or more agents.

As used herein, the term “conditioned cell culture medium” can refer to a medium in which cells have been grown. This means that the cells are grown for a sufficient time to secrete the factors that are effective to achieve any of the results or effects described herein. The term can also refer to a medium in which cells have been cultured so as to secrete factors into the medium. In some instances, cells can be grown through a sufficient number of cell divisions so as to produce effective amounts of such factors so that the medium so that the results or effects described herein are achieved. Cells can be removed from the medium by any of the known methods in the art including, but not limited to, centrifugation, filtration, immunodepletion (e.g., via tagged antibodies and magnetic columns), and FACS sorting.

As used herein, the terms “decrease” or “reduce” can mean to lower the effect or prevent it entirely, such as any of the effects described herein.

As used herein, the term “effective amount” can generally refer to an amount that provides the effect, e.g., effective to reduce, eliminate, or reverse lung remodeling. For example, an effective amount is an amount sufficient to effectuate a beneficial or desired clinical result. The effective amount can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations, which are routine in the art.

As used herein, the term “effective dose” can mean the same as “effective amount.”

As used herein, the term “effective route” can generally refer to a route that provides for delivery of an agent or composition to a desired compartment, system, or location. For example, an effective route is one through which an agent or composition can be administered to provide, at the desired site of action, an amount of the agent or composition sufficient to effectuate a beneficial or desired clinical result.

As used herein, the terms “increase” or “increasing” can mean to induce entirely where there was no pre-existing effect, or to increase the degree of the effect, such as any of the effects described herein.

As used herein, the term “isolated” can refer to a cell or cells which is/are not associated with one or more cells or one or more cellular components that is/are associated with the cell or cells in vivo. An “enriched population” means a relative increase in numbers of a desired cell relative to one or more other cell types in vivo or in primary culture. The term “isolated” can also indicate that the cells are removed from their natural tissue environment and are present at a higher concentration as compared to the normal tissue environment. This also can be expressed in terms of cell doublings, for example. A cell may have undergone 10, 20, 30, 40, or more doublings in vitro or ex vivo so that it is enriched compared to its original numbers in vivo or in its original tissue environment (e.g., bone marrow, peripheral blood, adipose tissue, etc.).

As used herein, the term “pharmaceutically-acceptable carrier” can refer to any pharmaceutically-acceptable medium for the cells disclosed herein. Such a medium may retain isotonicity, cell metabolism, pH, and the like. It is compatible with administration to a subject in vivo, and can be used, therefore, for cell delivery and treatment.

As used herein, the term “potency” can refer to the ability of cells (or conditioned medium from the cells), such as mesenchymal stem cells (MSCs) to achieve the various effects described herein. Accordingly, “potency” can refer to the effect(s) of MSCs at various levels, including, but not limited to: (1) reducing collagen deposition in the lungs of a subject; (2) increasing expression of a transcription factor (e.g., in an alveolar macrophage) of a subject; (3) decreasing expression of a cytokine (e.g., in an alveolar macrophage) of a subject (e.g., in a BAL sample); (4) decreasing expression of a toll-like receptor (TLR) (e.g., in an alveolar macrophage) of a subject; (5) decreasing mRNA synthesis of collagen I and/or collagen III in a subject; (6) decreasing the level of hyaluronic acid in a subject; (7) decreasing the number of macrophages in a subject (e.g., decreasing the percentage of macrophages in a BAL sample); (8) decreasing the number of eosinophils in a subject (e.g., decreasing the percentage of eosinophils in a BAL sample); (9) decreasing the number of polymorphonuclear leukocytes (PMNs) in a subject (e.g., decreasing the percentage of PMNs in a BAL sample); (10) decreasing systemic levels of an antibody; (11) decreasing weight loss; and (12) decreasing epithelial cell hyperplasia.

As used herein, the term “reduce” can mean to prevent as well as decrease. In the context of treatment, to “reduce” is to both prevent or ameliorate one or more clinical symptoms. A clinical symptom is one (or more) that has or will have, if left untreated, a negative impact on the quality of life (health) of the subject. This also applies to the biological effects, such as reducing collagen deposition in the lungs of a subject, reducing expression of a cytokine in an alveolar macrophage of a subject, reducing expression of a TLR in an alveolar macrophage of a subject, etc., the end result of which would be to ameliorate the deleterious effects of inflammation and lung remodeling.

As used herein, the term “selecting”, when used with reference to a cell with a desired level of potency, can mean identifying (as by assay), isolating, and expanding a cell. This could create a population that has a higher potency than the parent cell population from which the cell was isolated. To select a cell would include both an assay to determine if there is the desired effect and would also include obtaining that cell. The cell may naturally have the effect in that the cell was not incubated with or exposed to an agent that induces the effect. The cell may not be known to have the effect prior to conducting the assay. As the effects could depend on gene expression and/or secretion, one could also select on the basis of one or more of the genes that cause the effects. Selection could be from cells in a tissue. For example, in this case, cells would be isolated from a desired tissue, expanded in culture, selected for a desired effect, and the selected cells further expanded. Selection could also be from cells ex vivo, such as cells in culture. In this case, one or more of the cells in culture would be assayed for the effect and the cells obtained that have the effect could be further expanded. Cells could also be selected for enhanced effect. In this case, the cell population from which the enhanced cell is obtained already has the effect. Enhanced effectiveness means a higher average amount of the effect per cell than in the parent population. The parent population from which the enhanced cell is selected may be substantially homogeneous (the same cell type). One way to obtain such an enhanced cell from this population is to create single cells or cell pools and assay those cells or cell pools for the effect to obtain clones that naturally have the effect (as opposed to treating the cells with a modulator of the effect) and then expanding those cells that are naturally enhanced. However, cells may be treated with one or more agents that will enhance the effect of endogenous cellular pathways. Thus, substantially homogeneous populations may be treated to enhance modulation. If the population is not substantially homogeneous, then, it is preferable that the parental cell population to be treated contains at least 100 of the effective cell type in which enhanced effect is sought, more preferably at least 1,000 of the cells, and still more preferably, at least 10,000 of the cells. Following treatment, this sub-population can be recovered from the heterogeneous population by known cell selection techniques and further expanded if desired. Thus, desired levels of the effect may be those that are higher than the levels in a given preceding population. For example, cells that are put into primary culture from a tissue and expanded and isolated by culture conditions that are not specifically designed to have the effect, may provide a parent population. Such a parent population can be treated to enhance the average effect per cell or screened for a cell or cells within the population that express higher effect. Such cells can be expanded then to provide a population (e.g., a non-homogenous or heterogeneous population) with a higher (desired) effect. Selected MSCs can be autologous or non-autologous (e.g., allogeneic) and, in some instances, the phenotype of the selected MSCs (and thus their efficacy) can be changed by exposing the MSCs to different cues (e.g., growth factors) during culture.

As used herein, the term “subject” can be used interchangeably with the term “patient” and refer to any warm-blooded organism including, but not limited to, human beings, pigs, rats, mice, dogs, goats, sheep, horses, monkeys, apes, rabbits, cattle, etc.

As used herein, the term “therapeutically effective amount” can refer to the amount of an agent or composition determined to produce any therapeutic response in a subject. For example, effective therapeutic agents or compositions, such as those described herein may prolong the survivability of the patient, and/or inhibit overt clinical symptoms. Treatments that are therapeutically effective within the meaning of the term can include treatments that improve a subject's quality of life even if they do not improve the disease outcome per se. Such therapeutically effective amounts are readily ascertained by one of ordinary skill in the art. Thus, to “treat” means to deliver such an amount. Thus, treating can prevent or ameliorate any pathological symptoms of a lung remodeling disease. In some instances, the level of treatment will be relative to the given therapeutic dose and the health status of the subject.

“Treat,” “treating,” or “treatment” are used broadly in relation to the present disclosure and each such term encompasses, among others, preventing, ameliorating, inhibiting, or curing a deficiency, dysfunction, disease, or other deleterious process, including those that interfere with and/or result from a therapy.

As used herein, the terms “lung remodeling disease” or “LRD” can refer to a group of lung diseases affecting the alveoli and interstitium that result in progressive scarring and loss of function. LRDs can be classified into three categories: lung remodeling diseases associated with a decrease in lung volume or a restriction in lung expansion; lung remodeling diseases associated with an increase in lung volume; and lung remodeling diseases associated with constricted or blocked airways. Frequently, a LRD will feature both a pathological change in lung volume plus constricted or blocked airways. Further, a LRD can be characterized by a particular state of inflammation where, for example, a particular degree or type of observed inflammation skews the likelihood of developing a particular LRD (e.g., susceptibility to infection, airway injury, bleeding, etc.).

LRD may be caused by exposure to a number of toxins or pollutants including, but not limited to, antigenic allergies (e.g., dust mites, foods, dogs, cats, rodents, Hay fever, etc.); inorganic dust, such as silica or hard metal dust; asbestos fibers, noxious gases such as ammonia or chlorine gas; organic particles from various sources such as grain, sugar cane, and bird and animal droppings, moldy hay, and bacterial or fungal growths. LRD may also be caused by thoracic exposure to radiation or certain drugs such as chemotherapy drugs, various heart medications, certain psychiatric medications, and some antibiotics. In many cases, the cause of LRD is unknown and LRD is identified as idiopathic. Diagnosis of LRD is based on symptoms including dyspnea, nonproductive cough, fatigue, excessive mucus and/or sputum production, chest tightness, wheezing and low-grade fever; and generally involves blood tests, e.g., measurement of partial oxygen saturation of the blood; pulmonary function tests, e.g., measurement of total lung volume, residual volume, and vital capacity; and computed tomography (CT) scans of the thorax, e.g., to measure lung density and monitor lung remodeling.

LRDs can include diseases, disorders, and pathological conditions in which pathological or maladaptive remodeling occurs in the lung tissues, including bronchi, bronchioles, alveoli, airways, interstitium and pulmonary vasculature. LRD can include airway diseases like asthma and chronic bronchitis and lung tissue diseases like sarcoidosis. Other lung remodeling diseases include interstitial lungs diseases, e.g., hypersensitivity pneumonitis, collagen-vascular disease, eosinophilic granuloma, nonspecific interstitial pneumonitis, respiratory bronchiolitis-associated interstitial lung disease, desquamative interstitial pneumonitis, lymphocytic interstitial pneumonitis, acute interstitial pneumonitis. Numerous lung remodeling diseases are associated with occupational exposure to particulates including asbestosis, bauxite fibrosis, beryliosis, byssinosis, coal worker's pneumoconiosis, cork worker's lung, farmer's lung, mushroom worker's lung; Labrador Lung disease, pigeon breeder's lung, siderosis, silicosis, silo filler's disease and silicosiderosis. Certain lung remodeling diseases are associated with autoimmune diseases including, collagen-vascular diseases, e.g., systemic lupus erythematosus, rheumatoid arthritis (Caplan's syndrome), progressive systemic sclerosis, scleroderma, dermatomyositis, polymyositis, ankylosing spondylitis, Sjögren syndrome and mixed connective-tissue disease, or genetic diseases, e.g., cystic fibrosis and alpha-1 antitrypsin deficiency. Other lung remodeling diseases include bronchiectasis, emphysema, COPD; diseases associated with barotrauma and mechanical ventilation; high-altitude pulmonary edema, aspiration pneumonia, drug-induced pulmonary toxicity, lymphangioleiomyomatosis, lung transplantation rejection, e.g., bronchiolitis obliterans (BOS), pediatric and adult respiratory distress syndrome, hyperoxia-induced bronchopulmonary dysplasia, and remodeling associated with pulmonary hypertension and pulmonary venoocclusive disease.

Overview

The present disclosure relates generally to compositions and methods useful for treatment of lung remodeling diseases and, more particularly, to using mesenchymal stem cells having a desired potency to affect inflammation while at the same time decreasing remodeling and improving pulmonary function. The inventors of the present application investigated the efficacy of MSCs in a murine model of chronic asthma. It was discovered that MSCs: decrease weight loss, clinical score, epithelial cell hyperplasia, recruitment of BAL eosinophils, mucins, cytokines and systemic levels of IgE; decrease soluble and insoluble collagen levels; decrease type I and III collagen expression; increase expression of PPAR-γ; decrease expression of toll-like receptor 4 (TLR4); and decrease the level of hyaluronic acid. Based at least in part on these discoveries, and as discussed in more detail below, MSC therapy (e.g., MSCs per se or MSC-derived products, such as MSC conditioned cell culture medium) can: change the inflammatory profile of chronic asthma; decrease the presence of extracellular matrix (ECM); improve pulmonary function (e.g., by preventing and/or reversing lung remodeling); and provide an alternative therapeutic approach to the treatment of steroid-resistant chronic asthma.

Mesenchymal Stem Cells (MSCs)

One aspect of the present disclosure can include obtaining and using MSCs having a desired potency for one or more effects (discussed below) associated with reducing, preventing, and/or reversing pulmonary inflammation and/or lung remodeling. MSCs, or medicinal signaling cells (see Caplan, A I, Tissue Eng. Part A 16, 2415-2417), according to the present disclosure are derived from the embryonal mesoderm and can be isolated from many human and non-human sources, including adult bone marrow, peripheral blood, fat, placenta, and umbilical blood, among others. MSCs can differentiate, or have the ability to direct tissue differentiation (e.g., via paracrine effectors) into, many mesodermal tissues, including muscle, bone, cartilage, fat and tendon. In some instances, MSCs can be allogeneic or autologous. Methods for harvesting, isolating, and culturing (e.g., expanding) MSCs are known. See, for example, U.S. Pat. Nos. 5,226,914, 5,486,389, 5,827,735, 5,811,094, 5,736,396, 5,837,539, and 5,827,740, as well as Pittenger, M. et al, Science, 284:143-147 (1999), Dominici, M. et al., Cytotherapy, 8:315-317 (2006), and Dennis, J. E. et al., J. Orthop. Res. 22:735-741 (2004). For instance, medium for culturing MSCs can be serum-free or can contain protein supplements, such as platelet lysate or fetal calf serum.

Methods

Assessment Methods

Another aspect of the present disclosure can include an in vitro method for obtaining MSCs with a desired potency for one or more of the following: (1) reduce collagen deposition in the lungs of a subject; (2) increase expression of a transcription factor (e.g., in an alveolar macrophage) of a subject; (3) decrease expression of a cytokine (e.g., in an alveolar macrophage) of a subject; (4) decrease expression of a toll-like receptor (TLR) (e.g., in an alveolar macrophage) of a subject; (5) decrease mRNA synthesis of collagen I and/or collagen III in a subject; (6) decrease the level of hyaluronic acid in a subject; (7) decrease the number of macrophages in a subject (e.g., decreasing the percentage of macrophages in a BAL sample); (8) decrease the number of eosinophils in a subject (e.g., decreasing the percentage of eosinophils in a BAL sample); (9) decrease the number of PMNs in a subject (e.g., decreasing the percentage of PMNs in a BAL sample); (10) decrease systemic levels of an antibody; (11) decrease weight loss; and (12) decrease epithelial cell hyperplasia. The method can comprise assaying (e.g., in vitro) a preparation of MSCs for, and selecting, MSCs with a desired potency for one or more of effects (1)-(12).

The preparation of MSCs can include an isolated population of MSCs. For example, an isolated population of MSCs can be harvested from a subject (e.g., a human) prior to assaying for one or more of effects (1)-(12). The subject can be apparently healthy, suspected of having a LRD, or known to have a LRD.

In one example, reduced collagen deposition can include reduced insoluble collagen deposition and/or reduced soluble collagen deposition.

In another example, increased expression of a transcription factor can include increased expression of PPAR-γ.

In another example, decreased expression of a cytokine can include decreased expression of a cytokine that is indicative of a shaft away from Th2 cells to Th1 cells. For instance, decreased expression of a cytokine can include decreased expression of TNF-α, IL-13, and/or IL-5.

In another example, decreased expression of a TLR can include decreased expression of TLR-4.

In another example, decreased systemic levels of an antibody can include decreased systemic levels of IgE.

In another aspect, the method can include assaying (e.g., in vitro) a preparation of MSCs for and selecting MSCs with a desired potency for two or more of effects (1)-(12), three or more of effects (1)-(12), four or more of effects (1)-(12), five or more of effects (1)-(12), six or more of effects (1)-(12), seven or more of effects (1)-(12), eight or more of effects (1)-(12), nine or more of effects (1)-(12), ten or more of effects (1)-(12), eleven or more of effects (1)-(12), or all of effects (1)-(12).

Effects (1)-(12) can be assessed or assayed using one or a combination of conventional assays.

In one example, a 3T3 assay can be used to assess or measure the levels of soluble and/or insoluble collagen, as well as collagen gene expression. Examples of 3T3 assays are described by Chen, C. et al., Adv Exp Med Biol., 765:47-53 (2013) and Gloria, S. et al., Toxicol Lett. 228(2):111-26 (2014).

In another example, inflammation can be assessed by exposing epithelial cells or macrophages to LPS or TNF-α, culturing the exposed cells with or without MSCs, and then measuring secretion differences and/or gene expression of a target molecule or molecules (e.g., PPARγ, TLRs, etc.). Examples of assays for assessing inflammation are described by Bartosh, T J et al., Bio-protocol, 4(14):e1180 (2014) and Hye, J J et al., Int J Mol Sci., 14:17986-18001 (2013).

In another example, the ability of MSCs to decrease hyaluronic acid levels can be assessed by an ELISA-type assay, such as those described by Qu C. et al., Int J Biochem Cell Biol., 48:45-54 (2014) and Yu, F. et al., J Formos Med Assoc., 114(2):130-8 (2015).

In another example, the ability of MSCs to decrease PMN or eosinophil levels can be performed, for example, as described by Lai T S et al., Chin Med J. 128(3):361-7 (2015), Chimenti, L. et al., Eur Resp J. 40(4):939-48 (2012), Sun Y Q et al., Stem Cells, 30(12):2692-9 (2012), and Goodwin, M. et al., 29(7):1137-48 (2011).

Examples of assays for assessing the ability of MSCs to decrease systemic antibody levels are disclosed by Carrion, F A et al., Stem Cell Res Ther., 2:23 (2011) and Goodwin, M. et al., Stem Cells, 29(7):1137-48 (2011).

Therapeutic Methods

Another aspect of the present disclosure can include a method for treating a lung remodeling disease (LRD) in a subject. In some instances, treatment of an LRD can prevent, slow down, and/or reverse lung remodeling (e.g., by promoting resorption of scarred or dysfunctional lung tissue coupled with the regeneration of new, functional lung tissue). One step of the method can include assaying one or more MSCs (e.g., in a preparation of MSCs) for one or more of effects (1)-(12). A therapeutically effective amount of the one or more assayed MSCs found to have one or more of effects (1)-(12) can then be administered to the subject. Suitable formulations and compositions comprising assayed MSCs found to have one or more of effects (1)-(12) for administration are discussed below. Administered MSCs can exert their therapeutic effect(s) directly and/or indirectly. For example, administered MSCs can exert their therapeutic effect(s) by directly impacting the milieu of the lung. Additionally or alternatively, administered MSCs can exert their therapeutic effect(s) indirectly via a paracrine mechanism, such as by interaction with the host immune system (e.g., inducing inflammatory repair mechanisms).

Therapeutic methods of the present disclosure can prevent, reduce, stabilize, reverse or normalize various pathological features of LRD. Such pathological features include, but are not limited to, decreased lung volume, decreased potential air space, increased lung density, presence of remodeled lung tissue, increased septal thickness, decreased alveolarization, decreased pulmonary vascularization, increased medial wall thickness, decreased formation or presence of secondary septa, increased right ventricular systolic pressure (RVSP), increased right ventricular hypertrophy (RVH), decreased PaO₂ (arterial pressure of oxygen), increased lung volume, increased presence of PMNs in bronchoalveolar lavage (BAL) fluid and/or sputum, increased presence of eosinophils in BAL fluid and/or sputum, the presence of inflammation, and the presence of pulmonary edema.

Stabilizing a pathological feature of a LRD can be defined as reducing, slowing, or freezing the rate of decline of a pathological change in a lung tissue or in a lung parameter. In some instances, stabilization is the reduction in the rate at which a particular lung function is lost. In other instances, stabilization reduces the rate of a pathological change in a particular lung function by at least 5%, 10%, 15%, 20%, 25%, 30%, 35%, 40%, 45%, 50%, 75% or 100% compared to the expected rate of change based on a control population or a historical population. Stabilization can result in a halting of the rate of change in a particular lung function by the pathologic process, thereby halting the deterioration in the patient's lung function. Normalization can be the return to a normal range of a characteristic of a lung tissue or lung function. For example, normalization can result in the characteristic of the lung tissue or lung parameter being within the normal range or less than 5%, 10%, 15% or 20% away from the normal range. For instance, a patient with a LRD that had an initial lung density of −300 HU that increased to a lung density within the normal range of −800 to −900 HU after therapy according to the present disclosure would be classified as having normalized lung density. Similarly, a patient with a LRD that had an initial PaO₂ of 68% that increased to a PaO₂ of 81% after treatment according to the present disclosure would be classified as having a normalized PaO₂ as a value of 80% or above is within the normal range.

Lung remodeling may be measured by any method known to one of skill in the art. In some instances, lung remodeling can be measured by lung imaging from CT scans; more particularly, from HRCT. For example, lung density can be measured in Hounsfield Units (HU), and improvement in lung density as a result of the present methods can be measured as a decrease in measured HU (e.g., on the Hounsfield scale, lung zones with a density between −900 and −800 HU are typically considered within a normal aerated range, while those between −500 and −100 are poorly aerated and those between −100 and +100 are non-aerated). In other instances, lung remodeling can be measured by lung biopsy and histology. In further instances, lung remodeling can be measured as a change in potential airspace in lung tissue (e.g., a subject having a LRD can have decreased potential airspace in lung tissue relative to normal). In still other instances, lung remodeling can be measured by percentage of lung showing honeycomb changes or fibroblastic foci on lung images (e.g., a subject having a LRD may have an increased percentage of honeycomb change or fibroblastic foci in lung images). In other instances, lung remodeling can be measured by the presence or concentration of collagen I and/or collagen III fragments present in the blood of a subject (e.g., as a measure of lung scar turnover).

In some instances, the presence and extent of lung remodeling can be determined after measuring at least one functional lung parameter selected from the group consisting of vital capacity (VC), residual volume (RV), forced expiratory volume (FEV), forced vital capacity (FVC), forced expiratory flow (FEF), maximum flow (Vmax), peak expiratory flow rate (PEFR), inspiratory reserve volume (IRV), functional residual capacity (FRC), inspiratory capacity, total lung capacity (TLC), expiratory reserve volume (ERV), tidal volume and maximum voluntary ventilation (MVV). The patient's values can then be compared against standard lung function parameters for diagnosing the presence and extent of the LRD. Treatment with assayed MSCs found to have one or more of effects (1)-(12) can improve one or more functional lung parameters.

The measurement of a subject's PaO₂, diffusing capacity of the lung for CO (DLCO), or percent oxygen saturation of blood are also useful in assessing lung function because typically reduced values for functional lung parameters result in lower PaO₂, DLCO or percent oxygen saturation of blood values. Treatment with assayed MSCs found to have one or more of effects (1)-(12) can increase the PaO₂, DLCO or percent oxygen saturation of blood values of a patient with a LRD. For example, improved lung function increases or normalizes a patient's PaO₂.

Measurement of one or more lung function parameters, PaO₂, DLCO or percent oxygen saturation of blood values can be measured prior to treatment in a patient suspected of having a LRD and compared to one or more standard values to form the basis of a diagnosis. Treatment can be initiated if the measured lung function parameters, PaO₂, DLCO or percent oxygen saturation of blood values are appreciable outside a standard range of values, i.e., at least 5%, 10%, 15%, 20%, 25%, 30%, 40% or 50%. Further, treatment response can be monitored by measuring one or more lung function parameters of PaO₂, DLCO or percent oxygen saturation of blood values after treatment and comparing the measured lung function parameters to standard lung function parameters. An increase or normalization in one or more lung function parameters, PaO₂, DLCO or percent oxygen saturation of blood values indicate that the treatment is efficacious. The administration of assayed MSCs found to have one or more of effects (1)-(12) can be repeated if the measured lung function parameters, PaO₂, DLCO or percent oxygen saturation of blood values are below a desired range or appreciable outside of the standard range of values for the lung function parameters, i.e., at least 5%, 10%, 15%, 20%, 25%, 30%, 40% or 50%.

In another aspect, the presence and extent of a LRD can be measured by the cell count and cellular composition, such as BAL fluid and/or sputum. In some instances, the methods of the present disclosure can reduce or normalize the cellular and/or inflammatory profile of BAL fluid and/or sputum. In one example, the methods of the present disclosure can be used to treat a subject having an elevated percentage of PMNs in BAL fluid and/or sputum, with the methods of the present disclosure reducing the percentage of PMNs in BAL fluid and/or sputum. In another example, the methods of the present disclosure can be used to treat a subject having an elevated percentage of eosinophils in BAL fluid and/or sputum, with the methods of the present disclosure reducing the percentage of eosinophils in BAL fluid and/or sputum. The cell count and cellular composition of BAL fluid and/or sputum can be normalized over the treatment time course.

In another aspect, the presence and extent of a LRD can be measured by the presence and extent of pulmonary edema or cellular infiltration of lung tissue. In some instances, edema and/or cellular infiltration is an acute event, while in other instances the edema and/or cellular infiltration is a chronic event. Typically, cellular infiltration is associated with the proliferation of leukocytes. Thus, the methods of the present disclosure prevent, reduce or reverse pulmonary edema, cellular infiltration and/or cellular proliferation.

In some instances, therapeutic methods can be initiated upon diagnosis of a LRD in a subject. For example, a patient's lung density can be measured prior to treatment. The measured lung density can be compared to a standard lung density range and, if the lung density is denser than the standard lung density range, treatment with assayed MSCs found to have one or more of effects (1)-(12) can be initiated. Alternatively, a patient's lung density can be measured after treatment. The measured density can be compared to a standard lung density range and, if the lung density is denser than the standard density range, treatment can be repeated as necessary to normalize the patient's lung density.

In another aspect, the present disclosure can include methods and compositions for pre-treating a subject that has an increased probability of being afflicted with a LRM, thereby preventing or reducing the severity of a subsequent LRM.

In another aspect, the present disclosure can include a method of increasing the likelihood of survival in a subject having a LRD, the method comprising administering to the subject a therapeutically effective amount of assayed MSCs found to have one or more of effects (1)-(12), thereby increasing the likelihood of survival in the subject.

In another aspect, therapeutic methods of the present disclosure can be tailored to treat a particular LRD. Where it is known, for example, that a particular LRD (or symptom thereof) can be effectively treated by one or combination of effects (1)-(12), a preparation of MSCs can be assayed (as discussed above). Assayed MSCs found to have one or combination of desired effects (1)-(12) that is/are believed to be efficacious in treating the particular LRD can then be selected for administration to a subject having the particular LRD.

In one example, the present disclosure can include a method for treating a pulmonary fibrotic disease in a subject. The method can include the assaying one or more MSCs for one or more of the following effects: (1) reducing collagen deposition in the lungs of a subject; (2) increasing expression of a transcription factor (e.g., in an alveolar macrophage) of a subject; (3) decreasing expression of a cytokine (e.g., in an alveolar macrophage) of a subject; (4) decreasing expression of a toll-like receptor (TLR) (e.g., in an alveolar macrophage) of a subject; (5) decreasing mRNA synthesis of collagen I and/or collagen III in a subject; (6) decreasing the level of hyaluronic acid in a subject; (7) decreasing the number of macrophages in lung tissue of a subject (e.g., decreasing the percentage of macrophages in a BAL sample); (8) decreasing the number of eosinophils in lung tissue of a subject (e.g., decreasing the percentage of eosinophils in a BAL sample); (9) decreasing the number of polymorphonuclear leukocytes (PMNs) in lung tissue of a subject (e.g., decreasing the percentage of PMNs in a BAL sample); (10) decreasing systemic levels of an antibody; (11) decreasing weight loss in a subject; and (12) decreasing epithelial cell hyperplasia in a subject. A therapeutically effective amount of the one or more assayed MSCs found to have one or more of effects (1)-(12) can then be administered to the subject.

Generally speaking, fibrosis is a condition characterized by a deposition of extracellular matrix components in the skin and internal organs, such as the lungs. Pulmonary fibrosis, in particular, is a common pathologic reaction to non-specific post-inflammatory local fibrosis as well as specific processes that occur in interstitial pneumonias. Fibrotic changes cause functional dysfunction and are categorized as disease entities (e.g., interstitial pneumonia and bronchiectasis). Fibrosis of the lungs may occur in five distinct patterns: bronchial; interstitial; parenchymal; pleural; and vascular. The different patterns will, to a great extent, determine the type of functional disability, and may often coexist. For example, bronchial fibrosis will produce functional changes associated with diffuse obstructive emphysema, interstitial fibrosis will produce essentially diffusion disturbances, vascular fibrosis will produce pulmonary hypertension, and pleural fibrosis will produce some degree of ventilatory disturbance, as will advanced degrees of parenchymal fibrosis.

Pulmonary fibrosis is a major source of morbidity and mortality. Patients typically present with symptoms of cough and dyspnea; when the condition progresses, chronic respiratory failure often ensues. Although some forms of pulmonary fibrosis of known origin may have a better prognosis, idiopathic pulmonary fibrosis (IPF) is a progressive condition that rarely, if ever, remits spontaneously.

Accordingly, one aspect of the present disclosure can include treating a subject suffering from, or being suspected of suffering from, fibrosis of the lungs. Specific examples of conditions caused by, or associated with, fibrosis of the lungs, and that are treatable by the present disclosure, can include IPF, sarcoidosis, cystic fibrosis, fibrotic kidney disease, and dermatofibrosis. Treatment of a subject suffering from, or being suspected of suffering from, fibrosis of the lungs, can prevent, reduce, and/or reverse fibrosis in the lung tissue by, for example, preventing, reducing, and/or stabilizing deposition of collagen (e.g., soluble and insoluble collagen), preventing or reducing the presence of inflammatory molecules (e.g., cytokines, such as TNF-α), and/or preventing or reducing the presence of inflammatory cell types (e.g., PMNs, macrophages, eosinophils) in the lung tissue of the subject. In one example, reversing fibrosis can include preventing, stopping, or decreasing deposition of collagen (or ECM) in the lungs of a subject with an increase in healthy lung tissue, or replacement of remodeled lung tissue with healthy lung tissue, that is either concurrent with or subsequent to the prevention, stoppage, or decrease in collagen (or ECM) deposition. In another example, reversing fibrosis can include reversing (e.g., preventing, stopping, or decreasing) the synthesis of components associated with the fibrotic process, such as collagen. Measurements of fibrosis are focused on the amount of extracellular matrix components that are involved in the wound healing process that occurs to correct the damage associated with inflammation. Thus, according to the present disclosure, collagen synthesis can be assayed because collagen deposition produces scar, hyaluronan levels can be assayed because hyaluronan is part of the inflammatory process that connects scaring with inflammation, matrix metalloproteinases can be assayed and transcriptional regulators, such as PPARγ can be assayed because they are active proteases in stimulating scar formation and are regulated by transcriptional regulators, such PPARγ (which can also be assayed). In vitro, these effects or markers can be assessed using fibroblast cells since these cells are the main participants in the collagen deposition process (and thus ideal for the development of corresponding potency assays). In the Examples below, the in vivo chronic model of asthma is used because it advantageously has both pre-remodeling and enhanced extracellular deposition so that the in vivo effects can be monitored.

Administration Routes

A therapeutically effective amount of the one or more assayed MSCs found to have one or more effects (1)-(12) can be delivered to a subject via an effective route, e.g., any route that provides a suitable pharmacokinetic profile. In one example, assayed MSCs found to have one or more effects (1)-(12) can be administered intravenously (e.g., in a single bolus or infusion), as a particulate or aerosol directly to the lungs (e.g., via an inhaler), subcutaneously, intramuscularly, intra-arterially, or intraperitoneally.

Formulations

Assayed MSCs found to have one or more effects (1)-(12) can be delivered directly or in compositions containing excipients (e.g., as pharmaceutical compositions or medicaments), as is well known in the art. In one example, assayed MSCs found to have one or more effects (1)-(12) can be formulated as a locally administrable therapeutic composition. An effective amount of assayed MSCs found to have one or more effects (1)-(12) can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences; and Goodman and Gilman's The Pharmacological Basis of Therapeutics, 10^{th} Ed. (2001), Hardman, Limbird, and Gilman, eds. MacGraw Hill Intl.). Typically, assayed MSCs found to have one or more of effects (1)-(12) can be administered in an amount sufficient to provide therapeutic efficacy over the treatment time course. Therapeutic efficacy can be measured using any parameter provided herein, including improvement in any pathological feature of lung remodeling disease and/or improvement in lung function.

In some instances, the purity of the assayed MSCs found to have one or more of effects (1)-(12) (or conditioned medium therefrom) for administration to a subject can be about 100% (substantially homogeneous). In other instances, it can be 95% to 100%. In further instances, it can be 85% to 95%. Particularly, in the case of admixtures with other components, the percentage can be about 10%-15%, 15%-20%, 20%-25%, 25%-30%, 30%-35%, 35%-40%, 40%-45%, 45%-50%, 60%-70%, 70%-80%, 80%-90%, or 90%-95%. Or isolation/purity can be expressed in terms of cell doublings, where the MSCs have undergone, for example, 10-20, 20-30, 30-40, 40-50 or more cell doublings.

The choice of formulation for administering assayed MSCs found to have one or more of effects (1)-(12) (or conditioned medium therefrom) for a given application will depend on a variety of factors. Prominent among these will be the species of subject, the nature of the condition being treated, its state and distribution in the subject, the nature of other therapies and agents that are being administered, the optimum route for administration, survivability via the route, the dosing regimen, and other factors that will be apparent to those skilled in the art. For instance, the choice of suitable carriers and other additives will depend on the exact route of administration and the nature of the particular dosage form.

Final formulations of the aqueous suspension of MSCs/medium will typically involve adjusting the ionic strength of the suspension to isotonicity (i.e., about 0.1 to 0.2) and to physiological pH (i.e., about pH 6.8 to 7.5). The final formulation will also typically contain a fluid lubricant.

In some instances, cells/medium can be formulated in a unit dosage injectable form, such as a solution, suspension, or emulsion. Formulations suitable for injection of MSCs/medium typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of MSCs and optional additives, vehicles, and/or carrier in compositions to be administered in methods of the present disclosure. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

The MSCs can be suspended in an appropriate excipient in a concentration from about 0.01 to about 5×10⁶ cells/ml. Suitable excipients for injection solutions are those that are biologically and physiologically compatible with the MSCs and with the recipient, such as buffered saline solution or other suitable excipients. The composition for administration can be formulated, produced, and stored according to standard methods complying with proper sterility and stability.

Dosing

Doses for humans or other mammals can be determined without undue experimentation by the skilled artisan, from this disclosure, the documents cited herein, and the knowledge in the art. The dose of MSCs/medium appropriate to be used in accordance with various embodiments of the present disclosure will depend on numerous factors. The parameters that will determine optimal doses to be administered for primary and adjunctive therapy generally will include some or all of the following: the LRD being treated and its stage; the species of the subject, their health, gender, age, weight, and metabolic rate; the subject's immunocompetence; other therapies being administered; and expected potential complications from the subject's history or genotype. The parameters may also include: whether the MSCs are syngeneic, autologous or allogeneic; their potency (specific activity); the site and/or distribution that must be targeted for the MSCs/medium to be effective; and such characteristics of the site such as accessibility to MSCs/medium and/or engraftment of cells. Additional parameters include co-administration with other factors (such as growth factors and cytokines). The optimal dose in a given situation also will take into consideration the way in which the MSCs/medium are formulated, the way they are administered, and the degree to which the MSCs/medium will be localized at the target sites following administration.

In some instances, MSCs/medium may be administered in an initial dose, and thereafter maintained by further administration. MSCs/medium may be administered by one method initially, and thereafter administered by the same method or one or more different methods. The levels can be maintained by the ongoing administration of the MSCs/medium. Various embodiments administer the MSCs/medium either initially or to maintain their level in the subject or both by intravenous injection. In other instances, other forms of administration are used, dependent upon the patient's condition and other factors, discussed elsewhere herein.

MSCs/medium may be administered in many frequencies over a wide range of times. Generally, lengths of treatment will be proportional to the length of the disease process, the effectiveness of the therapies being applied, and the condition and response of the subject being treated.

Other Uses

Other uses are provided by knowledge of the biological mechanisms described in this application. One of these includes drug discovery. This aspect involves screening one or more compounds or agents for the ability to modulate the effects (1)-(12) of the MSCs. Such agents can include, but are not limited to, small organic molecules, antisense nucleic acids, siRNA DNA aptamers, peptides, antibodies, non-antibody proteins, cytokines, chemokines, and chemo-attractants. This would involve, first, developing an assay for the MSC's ability to effect (e.g., reduce) any of effects (1)-(12). Accordingly, the assay may be designed to be conducted in vivo or in vitro. Modulation assays could assess the activation state at any desired level, e.g., morphological, gene expression, functional, etc. An identified agent or compound can then be used to increase potency of the cells to achieve any of the above effects.

A further use for the invention is the establishment of cell banks to provide assayed MSCs found to have a potency for effects (1)-(12) for clinical administration. Generally, a fundamental part of this procedure is to provide MSCs that have a desired potency for administration in various therapeutic clinical settings (e.g., treating a LRD). Any of the same assays useful for drug discovery could also be applied to selecting MSCs for the bank as well as from the bank for administration. Accordingly, in such a procedure, the MSCs (or medium) would be assayed for the ability to achieve any of the above effects. Then, MSCs would be selected that have a desired potency for any of the above effects, and these cells would form the basis for creating a cell bank.

Compositions

The present disclosure is also directed to MSC populations with specific potencies for achieving any of the effects described herein (e.g., effects (1)-(12)). As described above, these populations are established by selecting for MSCs that have desired potency. These populations are used to make other compositions, for example, a cell bank comprising populations with specific desired potencies and pharmaceutical compositions containing a cell population with a specific desired potency.

The following Example is for the purpose of illustration only and is not intended to limit the scope of the claims, which are appended hereto.

### Example 1

Experiments were performed to determine the potential of MSCs to improve long-term outcome in chronic asthma.

Methods

Allergen Challenge Model of Allergic Asthma: OVA sensitization and challenge: BAlb/cJ mice, were purchased from the Jackson Laboratories (Bar Harbor, Me.) and sensitized by intra-peritoneal injections (100 μL) of 10 μg OVA emulsified in 1.5 mg of (Al(OH)₃) on day 0, day 14, and day 21 followed by exposure to 1% wt/vol OVA in PBS by aerosolization. Sham sensitization and challenges were carried out with sterile Al(OH)₃ in PBS. Animals were analyzed after 1 to 8 weeks of OVA challenge. Murine Cell Source: (a) Alveolar Macrophages: mice are injected subcutaneously with a lethal dose of ketamine (80 mg/kg) and xylazine (10 mg/kg), and bronchoalveolar lavage (BAL) is performed as known. The yield is usually around 5×10⁵/mouse. Total cell count and differential is evaluated on the lavage fluid using modified Wright Giemsa. BAL fluid is separated from the cell pellet and frozen for cytokine analysis. The pellet is saved for gene analysis and transcription factor analysis. Lung Pathology: lungs are either perfused through the left ventricle with sterile formalin or snap frozen for fixation sensitive markers. Cytokine Profiles: Th1/Th2-Luminex Multiplex: BAL fluid is centrifuged and stored at −70 C until evaluation for T-cell cytokines. The soluble microparticle based assays (Luminex-manifold) kits enables simultaneous multi-analyte detection and measurement of human cytokines and chemokines with limited volume and high sensitivity. hMSCs: 1×10⁶ hMSCs/100 μl PBS were given intravenously 3 days before sacrifice.

Results

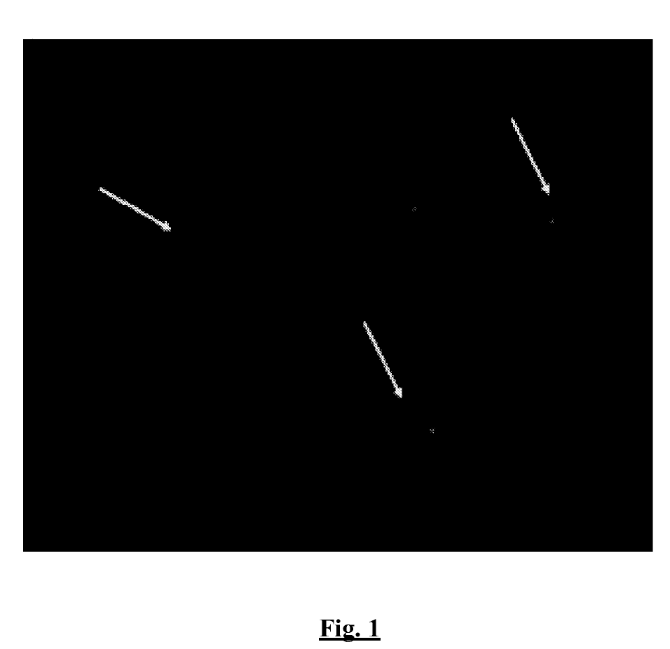

As shown in FIGS. 1-7B and described in more detail below, hMSCs: decreased weight loss, clinical score, epithelial cell hyperplasia, recruitment of BAL eosinophils, mucins, cytokines and systemic levels of IgE (P<0.05 for each); decreased soluble (28±6 to 13±9 mg collagen/lung) and insoluble (31±11 to 24±3 mg collagen/lung) collagen levels consistent with our ImagePro quantification; and decreased type I and III collagen expression by 54±11% (n=3, p=0.04) and 30±8% (n=3, p=0.02), respectively. More particularly, in FIG. 1, fluorescent-labeled hMSCs were given acute asthma mice after a 2 day challenge with ovalbumin. Animals were sacrificed after 7 days. Lungs were section and evaluated by fluorescent microscopy. FIG. 1 shows that labeled hMSCs localized to the lungs of the asthma mice.

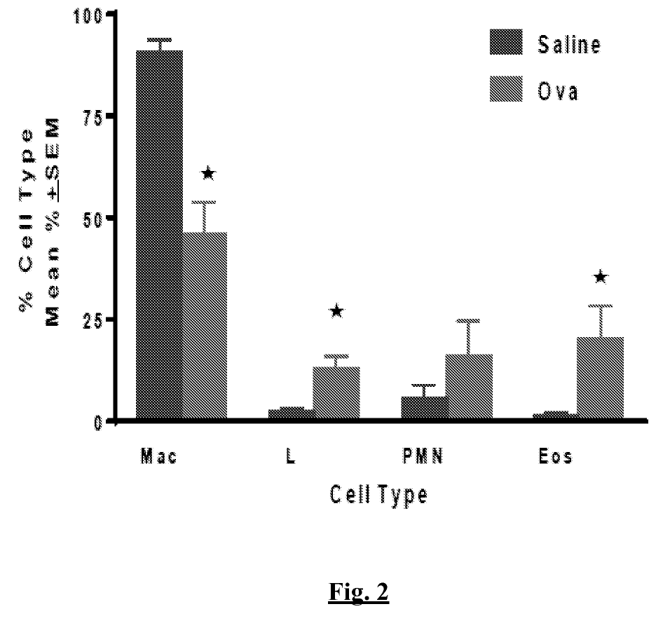

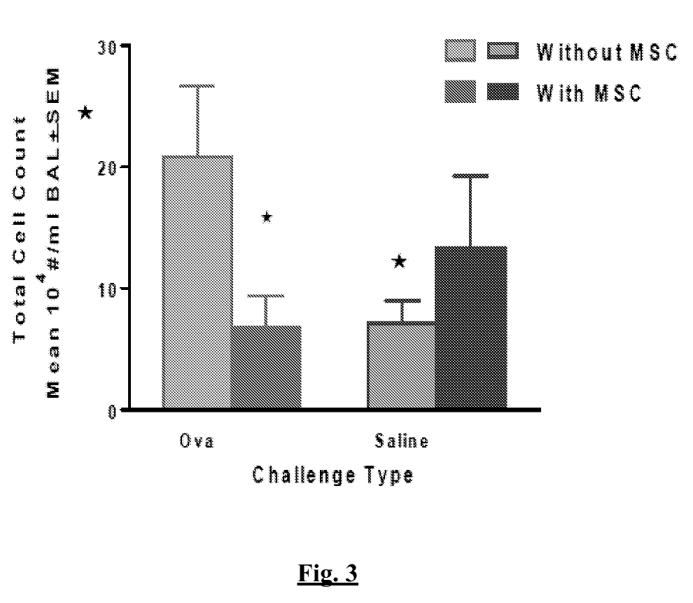

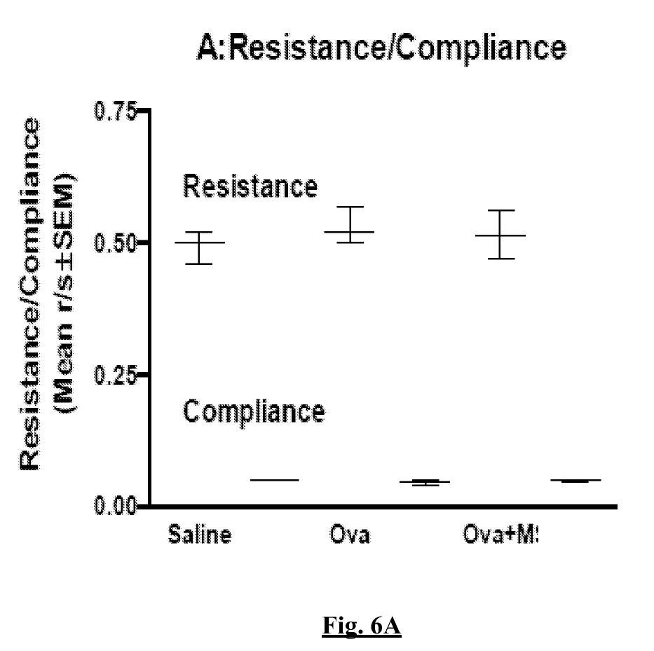

FIG. 2 shows cellular differentiation associated with chronic response. Animals were sensitized and rested for 14 days followed by 5 days of intranasal challenge like the acute model. On day 5, the mice are challenged three days of week for 4 weeks. 1 week prior to sacrifice, the animals received 1×10⁶/100 μl PBS hMSC. BAL was performed on every mouse along with cardiac puncture. Lungs were saved for pathology and hMSC localization (FIG. 3B). Decreased macrophages, increased eosinophils and neutrophils which are associated with and active chronic response (FIG. 5).

FIG. 3 shows that MSCs decrease total cell recruitment in the context of induced chronic lung inflammation. Mice challenged using the chronic asthma model were injected with hMSC. Mice were sacrificed and Lungs were evaluated for pathology and cellular differential. MSCs decreased total cell counts in BAL from mice challenged with ovalbumin (n=4, p<0.05). hMSC tended to induce cell recruitment in saline challenged mice.

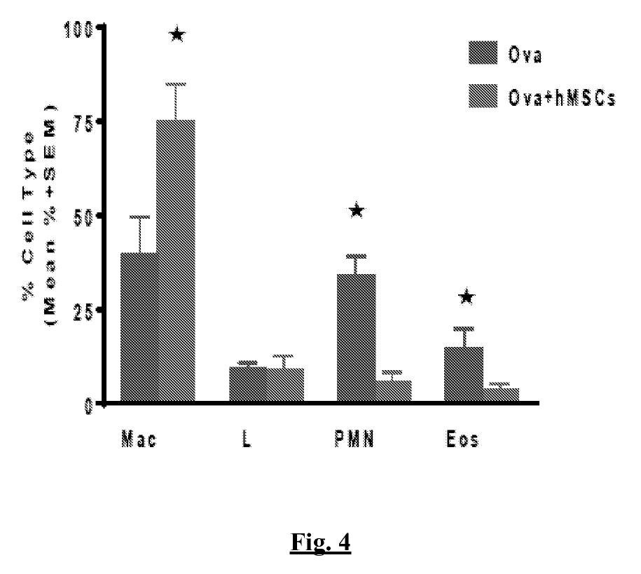

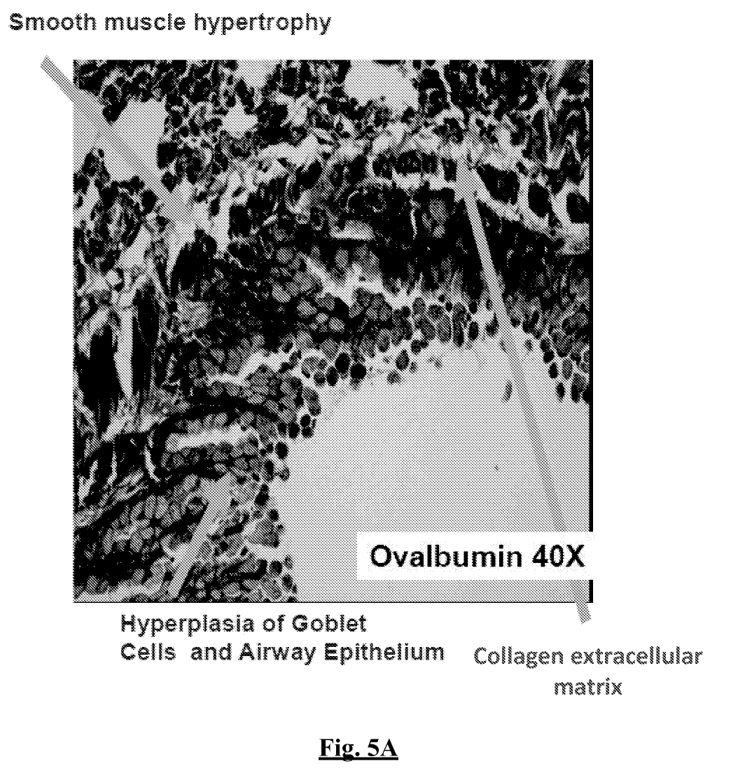

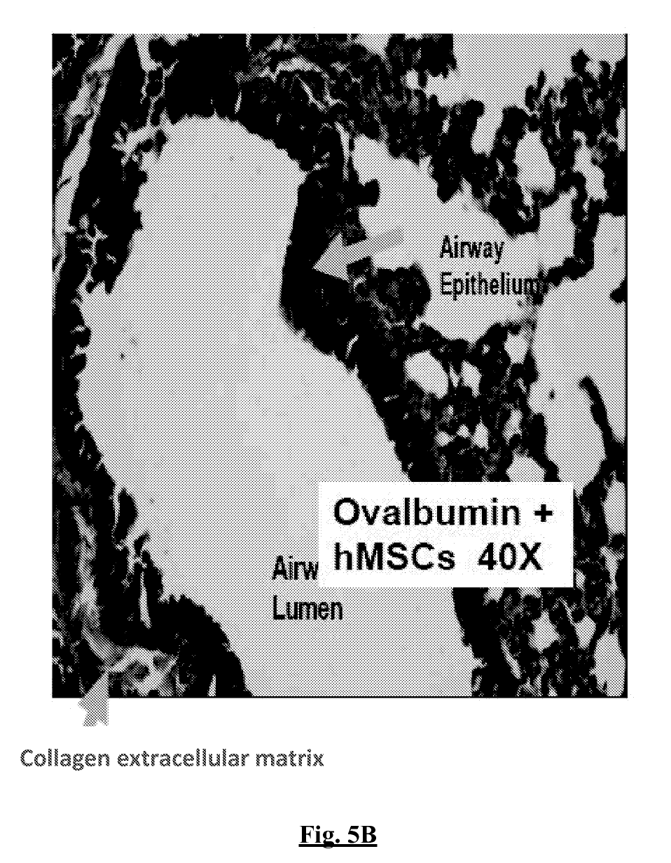

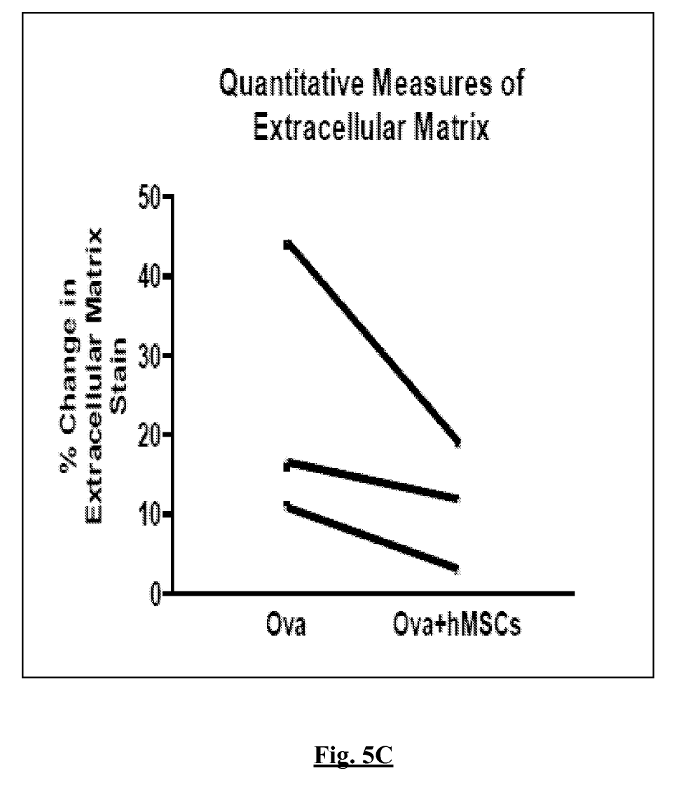

FIG. 4 shows that hMSC improve inflammation in chronic asthma mice. Chronic asthma mice treated with MSC had decreased macrophages and eosinophils while at the same presenting with elevated levels of neutrophils. This suggests that there is some MSC induced inflammation but also potential resolution of the asthma phenotype.

FIGS. 5A-C show that hMSC treatment decreased lung pathology associated with the chronic asthma model. Animals were sensitized and rested for 14 days followed by every other day challenge for 4 weeks. At the end of week 3, half of the mice were given hMSC intravenously. Mice were challenged for the last week. Lungs were removed after perfusion and sectioned for Trichrome staining. Ovalbumin model had significant bronchial hyperplasia and mucus (FIG. 5A), whereas animals treated with hMSCs had significant improved pathology (FIG. 5B). These images are representative of n=5. FIG. 5C shows the ImagePro quantified changes in ECM staining in three different experiments.

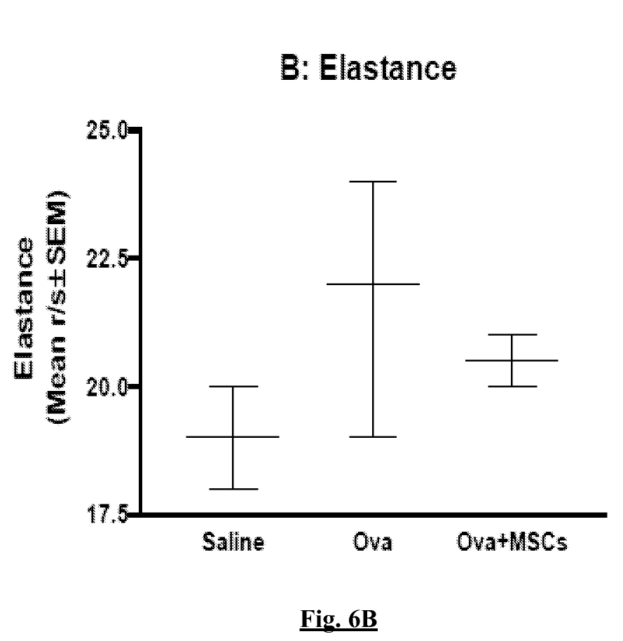

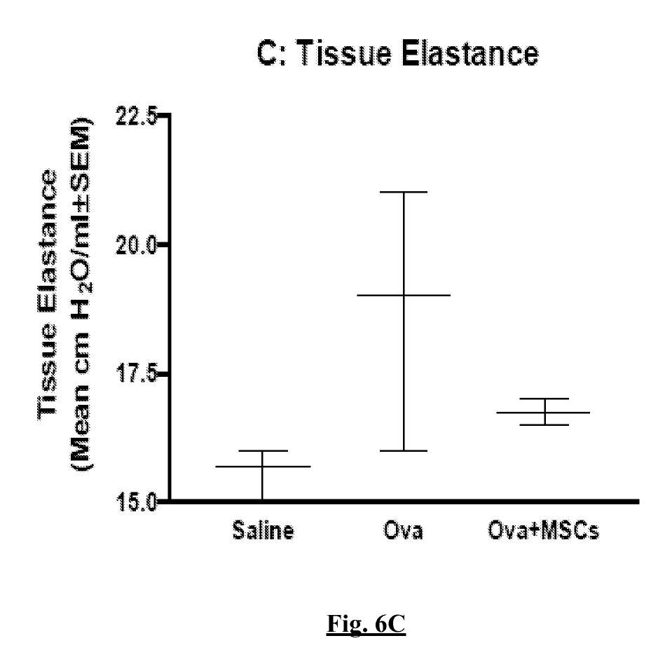

FIG. 6 shows plethysmography of the chronic ovalbumin model of asthma. Animals were anesthetized and evaluated for pulmonary function using whole body plethysmography. FIG. 6A shows resistance and compliance, FIG. 6B is airway elastance, and FIG. 6C is total tissue elastance. Resistance and compliance were not different between groups, but elastance and tissue elastance showed trends toward the controls when treated with MSCs.

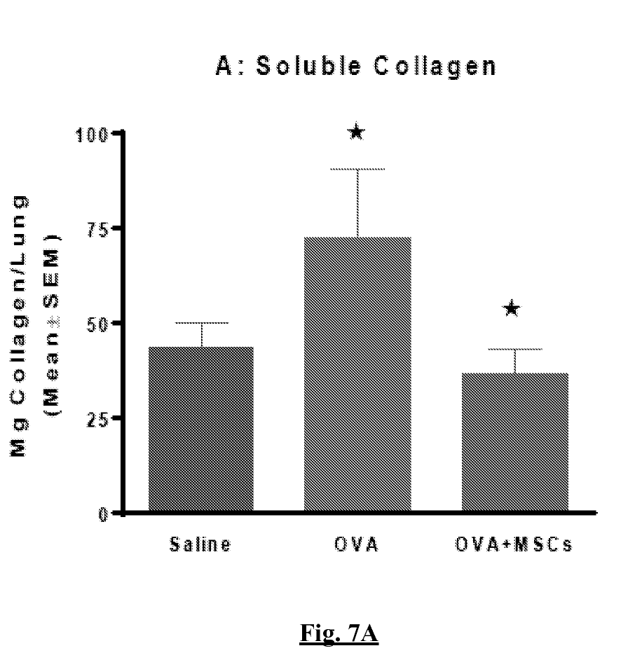

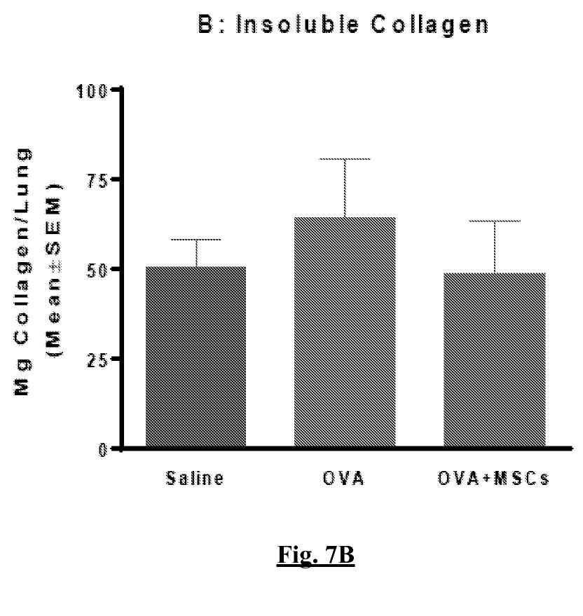

FIGS. 7A-B are a series of graphs showing soluble (FIG. 7A) and insoluble collagen (FIG. 7B) changes with hMSCs. Whole lungs were obtained from the 4-week chronic asthma model. Lungs were processed for either soluble (Sirius Red Assay) or insoluble (Hydroxyproline Assay). Each bar is 7 animals, but the results of a single chronic asthma study.

From the above description, those skilled in the art will perceive improvements, changes and modifications. Such improvements, changes, and modifications are within the skill of one in the art and are intended to be covered by the appended claims. All patents, patent applications, and publications cited herein are incorporated by reference in their entirety.

The following is claimed:

1. A method for treating a lung remodeling disease in a subject, the method comprising the steps of:
assaying one or more mesenchymal stem cells (MSCs) for one or more of the following effects: (1) reduce collagen deposition in the lungs of the subject; (2) increase expression of a transcription factor in an alveolar macrophage of the subject; (3) decrease expression of a cytokine in an alveolar macrophage of the subject; (4) decrease expression of a toll-like receptor (TLR) in an alveolar macrophage of the subject; (5) decrease mRNA synthesis of collagen 1 and/or collagen 3 in the subject; (6) decrease the level of hyaluronic acid in the subject; (7) decrease the number of macrophages in lung tissue of the subject; (8) decrease the number of eosinophils in lung tissue of the subject; (9) decrease the number of polymorphonuclear leukocytes (PMNs) in lung tissue of the subject; (10) decrease systemic levels of an antibody in the subject; (11) decrease weight loss in the subject; and (12) decrease epithelial cell hyperplasia in the subject; and
administering to the subject a therapeutically effective amount of the one or more assayed MSCs found to have one or more of effects (1)-(12).

2. The method of claim 1, wherein the transcription factor is PPAR-γ.

3. The method of claim 1, wherein the cytokine is TNF-α.

4. The method of claim 1, wherein the TLR is TLR-4.

5. The method of claim 1, wherein effect (1) further includes reduce insoluble and soluble collagen deposition.

6. The method of claim 1, wherein the MSCs are allogeneic or autologous.

7. The method of claim 1, wherein the lung remodeling disease is a pulmonary fibrotic disease.

8. The method of claim 1, wherein the lung remodeling disease is chronic asthma.

9. The method of claim 1, wherein the administered MSCs exert a therapeutic effect by directly impacting the milieu of the lung.

10. The method of claim 1, wherein the administered MSCs exert a therapeutic effect by via a paracrine mechanism.

11. An in vitro method for obtaining MSCs with a desired potency for one or more of the following: (1) reduce collagen deposition in the lungs of the subject; (2) increase expression of a transcription factor in an alveolar macrophage of the subject; (3) decrease expression of a cytokine in an alveolar macrophage of the subject; (4) decrease expression of a toll-like receptor (TLR) in an alveolar macrophage of the subject; (5) decrease mRNA synthesis of collagen 1 and/or collagen 3 in the subject; (6) decrease the level of hyaluronic acid in the subject; (7) decrease the number of macrophages in lung tissue of the subject; (8) decrease the number of eosinophils in lung tissue of the subject; (9) decrease the number of PMNs in lung tissue of the subject; (10) decrease systemic levels of an antibody in the subject; (11) decrease weight loss in the subject; and (12) decrease epithelial cell hyperplasia in the subject, the method comprising assaying a preparation of MSCs for and selecting MSCs with a desired potency for one or more of the effects (1)-(12) above.

12. The method of claim 11, wherein the transcription factor is PPAR-γ.

13. The method of claim 11, wherein the cytokine is TNF-α.

14. The method of claim 11, wherein the TLR is TLR-4.

15. The method of claim 11, wherein effect (1) further includes reduce insoluble and soluble collagen deposition.

16. A method to construct a cell bank, the method comprising expanding and storing the selected cells of claim 11 for future administration to a subject.

17. A method for drug discovery, the method comprising contacting the selected cells of claim 11 with an agent to assess the effect of the agent on the ability of the MSCs to effect any of effects (1)-(12).

18. A composition comprising MSCs assayed and selected for a desired potency to achieve one or more of the following effects: (1) reduce collagen deposition in the lungs of the subject; (2) increase expression of a transcription factor in an alveolar macrophage of the subject; (3) decrease expression of a cytokine in an alveolar macrophage of the subject; (4) decrease expression of a toll-like receptor (TLR) in an alveolar macrophage of the subject; (5) decrease mRNA synthesis of collagen 1 and/or collagen 3 in the subject; (6) decrease the level of hyaluronic acid in the subject; (7) decrease the number of macrophages in lung tissue of the subject; (8) decrease the number of eosinophils in lung tissue of the subject; (9) decrease the number of PMNs in lung tissue of the subject; (10) decrease systemic levels of an antibody in the subject; (11) decrease weight loss in the subject; and (12) decrease epithelial cell hyperplasia in the subject.

19. The composition of claim 18, wherein the MSCs are allogeneic or autologous.

20. The composition of claim 18, being formulated as a locally administrable therapeutic composition.
